(19) United States
(12) Patent Application Publication
(10) Pub. No.: US 2018/0042835 A1
(43) Pub. Date: Feb. 15, 2018

(54) COSMETIC PREPARATIONS WITH LOW TEXTILE ADHESION

(71) Applicant: Henkel AG & Co. KGaA, Duesseldorf (DE)

(72) Inventors: Thomas Doering, Dormagen (DE); Stefanie Schmitz, Moenchengladbach (DE)

(73) Assignee: Henkel AG & Co. KGaA, Duesseldorf (DE)

(21) Appl. No.: 15/554,435

(22) PCT Filed: Mar. 3, 2016
(86) PCT No.: PCT/EP2016/054493

§ 371 Date: Aug. 30, 2017

(30) Foreign Application Priority Data
Mar. 26, 2015 (DE) 10 2015 205 477.2

(51) Int. Cl. A61K 8/891; A61K 8/58; A61K 8/37; A61Q 15/00; A61K 8/26

(52) U.S. Cl. CPC A61K 8/891; A61Q 15/00; A61K 8/26; A61K 8/37; A61K 8/585; A61K 2800/24

(57) ABSTRACT

The present disclosure relates to cosmetic compositions for personal care. In one example, a cosmetic preparation includes, based on the total weight of the composition, an oil mixture in a total quantity of about 40 to about 99 wt. %. The oil mixture includes a) at least one ester, which is formed from at least one linear or branched, saturated or unsaturated mono-, di-, or tricarboxylic acid with 3 to 30 carbon atoms, wherein the acid can optionally contain one or more hydroxyl groups, and at least one linear or branched, saturated or unsaturated alcohol with 1 to 30 carbon atoms, b) at least cyclic polydimethylsiloxane, and c) at least one linear polydimethylsiloxane which has a kinematic viscosity of less than about 20 cSt at 25° C.

### CROSS-REFERENCE TO RELATED APPLICATION

This application is a U.S. National-Stage entry under 35 U.S.C. § 371 based on International Application No. PCT/EP2016/054493, filed Mar. 3, 2016 which was published under PCT Article 21(2) and which claims priority to German Application No. 10 2015 205 477.2, filed Mar. 26, 2015, which are all hereby incorporated in their entirety by reference.

### TECHNICAL FIELD

The present disclosure relates to cosmetic preparations for personal care, which contain a special oil mixture and are particularly suited for production as an antiperspirant.

### BACKGROUND

Washing, cleaning and caring for the human body is an important basic need, and cosmetics manufacturers are always trying to meet the constantly changing and evolving requirements of consumers by supplying innovative and/or improved products. Cosmetic care products often contain a high proportion of one or more oils and can cause stains to form upon contact with textiles. In particular in combination with other ingredients and active agents contained in care products, such as dyes and/or antiperspirant (aluminum) salts, textile stains can be particularly stubborn and difficult to wash out.

Makeup stains around the collar or neckline and/or white residues in the underarm area of textiles are typical examples of textile stains that are difficult to wash out. White and/or yellow stains in the underarm area of textiles can result from the regular use of antiperspirants. These stains are likely to be caused by the formation of initially insoluble aluminum compounds on and within the textile fiber. The yellow coloration usually takes effect with a time delay and is at least partially caused by the oxidation of unsaturated fatty acids that are present in the form of insoluble aluminum salts. In addition, various other factors can interact in an unexpected way and, for example, cause pronounced yellow stains to form on textiles, depending on the choice of perfume oil, detergent and/or the individual quantity and composition of sweat.

The interaction between detergents and antiperspirant active ingredients produces insoluble compounds, which can accumulate on a textile. These insoluble compounds form white, hard residues, which do not usually show on the textile until after several soiling and cleaning cycles. These white residues are not soluble in water and cannot be removed in a standard washing process. They are particularly visible on light or dark-colored textiles. The skillful selection of additives considerably reduces or delays the formation of these insoluble residues.

Oils with a high refractive index, which conceal the white residues, are described as a suitable additive in antiperspirants. However, oils with a high refractive index tend to accumulate, in particular on cotton, and form dark fatty or oily stains, which, among other things, can have a negative impact on the feel of textiles in the soiled area.

By using mainly volatile oils such as cyclopentasiloxane in antiperspirants, it was possible, in turn, to largely prevent the emergence of wet, dark oil stains on textiles, but not the emergence of the white stains caused by (aluminum) salt residues.

A further disadvantage of using only volatile oils in cosmetic preparations is that volatile oils often do not have satisfactory skin caring properties and that they can result in a dusty, unfocused spray jet in aerosol applications, in particular in high concentrations. Consequently, there is also a need for cosmetic products for personal care that leave fewer or no stains on textiles, or only stains that can be easily and thoroughly removed through washing.

### BRIEF SUMMARY

Cosmetic preparations for personal care are provided herein. In an exemplary embodiment, a cosmetic preparation for personal care includes, relative to the total weight of the cosmetic preparation, an oil mixture in a total quantity of from about 40 to about 99 wt. %. The oil mixture includes a) at least one ester that is formed from at least one linear or branched, saturated or unsaturated mono-, di- or tricarboxylic acid with from 3 to 30 carbon atoms, which comprises one or more hydroxyl groups, and at least one linear or branched, saturated or unsaturated alcohol with 1 to 30 carbon atoms, b) at least one cyclic polydimethylsiloxane, and c) at least one linear polydimethylsiloxane that has a kinematic viscosity of less than about 20 cSt at 25° C.

In another exemplary embodiment, a cosmetic antiperspirant preparation includes a) at least one ester a) that is formed from at least one linear or branched, saturated or unsaturated mono-, di- or tricarboxylic acid with from 3 to 30 carbon atoms, which includes one or more hydroxyl groups, and at least one linear or branched, saturated or unsaturated alcohol with from 1 to 30 carbon atoms, with a weight proportion of from about 10 to about 50 wt. % of the total weight of the preparation, b) at least one cyclic polydimethylsiloxane b) with a weight proportion of from about 5 to about 30 wt. % of the total weight of the preparation, c) at least one linear polydimethylsiloxane c) that has a kinematic viscosity of less than about 20 cSt at 25° C., with a weight proportion of from about 1 to about 20 wt. % of the total weight of the preparation, and d) at least one antiperspirant aluminum salt d) with a weight proportion of from about 10 to about 40 wt. % of the total weight of the preparation.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The objective of this present disclosure was to provide cosmetic products for personal care that have a high content of skin caring oils, which ensure good adhesion to the skin or good absorption into the skin. At the same time, the cosmetic products should have a low degree of adhesion to textiles and also cause as little stain formation as possible when they interact with other ingredients.

Another objective was to formulate, above all, oil-based, highly skin-compatible antiperspirants so that they show fewer or no dark oil stains and/or white to yellow stains, even after regular use.

Surprisingly, it was then discovered that the above-mentioned objectives can be solved to an exceptional degree using cosmetic preparations for personal care, containing a special mixture of various oils.

The oil mixtures can be easily incorporated into a wide variety of cosmetic care preparations and can even serve as the primary basis of cosmetic preparations. They have excellent skin care effects and are very easily washed out of textiles, especially cotton. Furthermore, the oil mixtures are extremely effective in the concealment/reduction/prevention of white aluminum salt residues when used in antiperspirants.

Accordingly, a first subject matter of the present disclosure is a cosmetic preparation for body care, which (relative to the total weight of the preparation in each case) contains an oil mixture in a total quantity of from about 40 to about 99 wt. %, wherein the oil mixture contains

a) at least one ester that is formed from at least one linear or branched, saturated or unsaturated mono-, di- or tricarboxylic acid with 3 to 30 carbon atoms, which can contain one or more hydroxyl groups, and at least one linear or branched, saturated or unsaturated alcohol with from about 1 to about 30 carbon atoms.

b) at least one cyclic polydimethylsiloxane, and

c) at least one linear polydimethylsiloxane that has a kinematic viscosity of less than 20 cSt at 25° C.

Suitable cosmetic preparations for personal care are skin treatment preparations preferred as contemplated herein, which have a high content of hydrophobic substances (oils), such as skin creams, makeup, lipsticks, other decorative cosmetic articles such as eye shadow, peelings, skin lotions, deodorants, antiperspirants, hair oils, styling products and/or pomades.

Cosmetic preparations that contain the above-mentioned oil mixture with a weight proportion of from about 40 to about 99 wt. %, preferably from about 45 to about 99 wt. %, more preferably from about 50 to about 99 wt. %, most preferably from about 55 to about 99 wt. % and especially preferably from about 60 to about 99 wt. %. Cosmetic preparations that contain liquid oils a), b) and c) in the oil mixture are most preferable.

As contemplated herein, a liquid oil is a liquid substance that is miscible with double-distilled water to less than about 1 wt. % under normal conditions. All information about the physical states of substances (solid, liquid, gaseous) in this application refers to normal conditions. In the sense of this application, “normal conditions” constitute a temperature of 20° C. and a pressure of 1013.25 mbar.

It was discovered that the use of a mixture of esters a), in particular a mixture of different esters a) with a high refractive index, is especially well tolerated by the skin and, in combination with cyclic polydimethylsiloxanes and low-viscous linear polydimethylsiloxanes, is very easily washed out of textiles.

To the extent required by the present disclosure, “high refractive index” are esters a), which have a refractive index (nD) of > about 1,420, preferably > about 1,425 and most preferably > about 1,430 at 20° C.

Oil mixtures that contain at least one ester from a first group a-1) and at least one ester from a second group of esters a-2) are particularly suitable.

Oil mixtures that contain at least two different esters from group a-1) and at least one ester from a second group of esters a-2) are most preferable as such a combination of esters not only had a positive effect on the ease of washing out the cosmetic preparations, but also enabled white powder residues on textiles to be particularly well concealed, especially in antiperspirants.

Particularly suitable esters from group a-1) are preferably selected from esters derived from at least one branched alcohol with from 4 to 20, preferably with from 5 to 18 and most preferably with from 6 to 16 carbon atoms and at least one linear or branched, saturated or unsaturated carboxylic acid with from 8 to 24, preferably with from 10 to 22 and most preferably with from 12 to 20 carbon atoms.

Examples of preferred esters a-1) are 2-ethylhexyl laurate, 2-ethylhexyl myristate, 2-ethylhexyl palmitate, 2-ethylhexyl cocoate, 2-ethylhexyl stearate, 2-ethylhexyl isostearate, hexyldecyl laurate, hexyldecyl stearate, isopropyl myristate, isopropyl palmitate, isopropyl stearate, isooctyl stearate, isononyl isononanoate, isononyl stearate, isotridecyl nonanoate, 2-octyldodecyl palmitate, isocetyl stearate and mixtures of these esters.

Most preferred esters a-1) are 2-ethylhexyl laurate, 2-ethylhexyl myristate, 2-ethylhexyl palmitate, 2-ethylhexyl stearate, 2-ethylhexyl isostearate, isopropyl myristate and/or isopropyl palmitate. Among the above-mentioned esters a-1), those that are liquid under normal conditions and preferably have a boiling point > about 120° C., more preferably > about 130° C. and most preferably > about 140° C. are especially preferred. 2-ethylhexyl palmitate and isopropyl myristate are especially preferred.

Particularly suitable esters a-2) are all optical esters that include at least one C2-C7 mono-, di- or tricarboxylic acid, which may contain one or more hydroxyl groups, and at least one linear or branched, saturated or unsaturated alcohol with from 1 to 10, preferably with from 1 to 7 and more preferably with from 1 to 4 carbon atoms.

More preferable esters a-2) are selected from methyl-, ethyl-, n-propyl-, isopropyl-, n-butyl-, 2-ubtyl- or tertiary butyl esters of glycolic acid, lactic acid, malic acid, tartaric acid, citric acid, oxalic acid, malonic acid, succinic acid, glutaric acid and/or adipic acid. Most preferred esters a-2) are methyl-, ethyl- and isopropyl esters of lactic acid, tartaric acid, oxalic acid, malonic acid, succinic acid, adipic acid and citric acid, such as ethyl lactate, dimethyl tartrate, diethyl tartrate, dimethyl oxalate, diethyl oxalate, dimethyl malonate, diethyl malonate, dimethyl succinate, diethyl succinate, dimethyl adipate, diethyl adipate, diisopropyl adipate, trimethyl citrate and triethyl citrate.

Among the above-mentioned esters a-2), those esters that are liquid under normal conditions and preferably have a boiling point > about 150° C., more preferably > about 175° C., most preferably > about 200° C. and especially preferably > about 225° C., are most preferred. Triethyl citrate is especially preferred.

In a first preferred embodiment of the present disclosure, cosmetic preparations are exemplified as contemplated herein in that the oil mixture contains

at least one ester a-1) selected from 2-ethylhexyl laurate, 2-ethylhexyl myristate, 2-ethylhexyl palmitate, 2-ethylhexyl cocoate, 2-ethylhexyl stearate, 2-ethylhexyl isostearate, hexyldecyl laurate, hexyldecyl stearate, isopropyl myristate, isopropyl palmitate, isopropyl stearate, isooctyl stearate, isononyl isononanoate, isononyl stearate, isotridecyl nonanoate, 2-octyldodecyl palmitate, isocetyl stearate and/or mixtures of these esters and

at least one ester a-2), selected from methyl-, ethyl-, n-propyl-, isopropyl-, n-butyl-, 2-ubtyl- or tertiary butyl esters of glycolic acid, lactic acid, malic acid, tartaric acid, citric acid, oxalic acid, malonic acid, succinic acid, glutaric acid and/or adipic acid.

In a second preferred embodiment, cosmetic preparations are exemplified as contemplated herein in that the oil mixture contains at least two different esters from group a-1).

In this embodiment, oil mixtures that contain 2-ethylhexyl palmitate and isopropyl myristate as ester a-1) are most preferred due to their excellent ability to conceal white powder residues (such as aluminum salts).

In a third preferred embodiment, cosmetic preparations are exemplified as contemplated herein in that the oil mixture contains a methyl or ethyl ester of tartaric acid, citric acid and/or oxalic acid, preferably a methyl or ethyl ester of citric acid and most preferably triethyl citrate, as ester a-2).

A further preferred embodiment of the present disclosure is exemplified in that the cosmetic preparations contain

2-ethylhexyl palmitate,

isopropyl myristate and

triethyl citrate as ester a).

A proportion of cyclic polydimethylsiloxanes is required in cosmetic preparations as contemplated herein in order to reduce the tendency of ester oils a) to form dark oil stains on textiles, in particular on cotton textiles, and to increase the ease of washing out the preparations.

Particularly suitable cyclic polydimethylsiloxanes b) are highly volatile and have a vapor pressure of < about 2.5 hPa, more preferably < about 2.0 hPa, most preferably < about 1.5 hPa and especially preferably < about 1.0 hPa at 20° C.

Examples of particularly suitable cyclic polydimethylsiloxanes c) include hexamethylcyclotrisiloxane, octamethylcyclotetrasiloxane, decamethylcyclopentasiloxane and/or dodecamethylcyclohexasiloxane.

Decamethylcyclopentasiloxane is especially preferred.

In a fourth preferred embodiment, cosmetic preparations are exemplified as contemplated herein in that the oil mixture contains at least one cyclic polydimethylsiloxane b) that is selected from hexamethylcyclotrisiloxane, octamethylcyclotetrasiloxane, decamethylcyclopentasiloxane and/or dodecamethylcyclohexasiloxane, preferably decamethylcyclopentasiloxane.

As a third mandatory ingredient, the preparations as contemplated herein contain at least one linear polydimethylsiloxane, which has a kinematic viscosity of less than about 20 cSt at 25° C., as an integral part of the oil mixture.

The addition of low-viscosity linear polydimethylsiloxanes to the oil mixtures gives the oil mixtures and the preparations as contemplated herein particularly beneficial properties in terms of application technology. In particular, this includes the rheological properties of the oil mixtures or the cosmetic preparations as contemplated herein, such as spreading behavior. Furthermore, the addition of linear polydimethylsiloxanes, which have a kinematic viscosity of less than about 20 cSt at 25° C., to the oil mixtures enhances the ease of washing out both oil stains on textiles, which can be caused by ester oils with a high refractive index, and white stains on textiles caused by the separation of aluminum salts from antiperspirants.

Particularly suitable linear polydimethylsiloxanes c) preferably have a viscosity (determined at 25° C. according to Brookfield) in the range of from about 2 to about 10 mPas, most preferably from about 4 to about 8 mPas. The kinematic viscosity of most preferred linear polydimethylsiloxanes c) is preferably less than about 20 cSt at 25° C., more preferably less than about 15 cSt and most preferably less than about 10 cSt.

Particularly suitable linear polydimethylsiloxanes c) are commercially available, such as under the trade name Xiameter® PMX 200 Sil Fluid 5 CS produced by Dow Corning. In a fifth preferred embodiment cosmetic preparations exemplified as contemplated herein in that the oil mixture contains at least one linear polydimethylsiloxane c) that has at 25° C. a kinematic viscosity of less than about 15 cSt, preferably less than about 10 cSt.

The previously described objectives are solved in a particularly satisfactory way if oils a), b) and c) are in particular proportions in the oil mixture.

It was discovered that the adhesion of cosmetic preparations as contemplated herein to textiles is particularly low when the oil mixture contains an amount of ester oils a) that is slightly larger than the total amount of silicon oils b) and c).

Furthermore, in particular proportions, silicon oils b) and c) contribute to the reduced adhesion of ester oils on textiles and significantly increase the ease of washing out the cosmetic preparations.

In addition, it was discovered that an oil mixture that contains oils a), b) and c) in particular proportions can be very successfully integrated into a wide variety of cosmetic preparations. The corresponding cosmetic preparations absorb very well into the skin and nourish it.

In a sixth preferred embodiment, cosmetic preparations are exemplified as contemplated herein in that the oil mixture (relative to the total weight relative to its total weight) contains

from about 35 to about 70 wt. % of at least one ester a), preferably

from about 40 to about 60 wt. % of at least two different esters from group of esters a-1), and

from about 1 to about 6 wt. % of at least one ester from group of esters a-2),

from about 20 to about 40 wt. % of at least one cyclic polydimethylsiloxane b) and

from about 5 to about 20 wt. % of at least one linear polydimethylsiloxane c) that has a kinematic viscosity of less than about 20 cSt at 25° C.

In this embodiment, it is most preferred if the oil mixture (relative to its total weight) contains

from about 36- about 46 wt. % of 2-ethylhexyl palmitate (ester a-1)),

about 15 wt. % of isopropyl myristate (ester a-1)),

from about 1- about 6 wt. % of triethyl citrate (ester a-2)),

from about 30- about 40 wt. % of cyclopentasiloxane (cyclic polydimethylsiloxane b)) and

from about 5- about 15 wt. % of dimethicone, 5 cSt.

As already stated, oil mixtures that contain ingredients a), b) and c) significantly improve the ease of washing out cosmetic preparations based on oil mixtures. In addition, the oil mixtures have excellent rheological properties and also give the cosmetic preparations, in which they can be incorporated, excellent rheological properties, in particular excellent spreading behavior.

Through a careful combination of oils a), b) and c), oil mixtures with particular refractive indices can be produced that have a very low adhesion to textiles and are very easy to wash out of textiles.

“Spreading” refers to the ability of a liquid substance to spread out after contact with a surface. The spreadability value is used as measure of the spreading behavior of a liquid substance on a surface. The spreadability value can be determined according to a procedure known in the state of technology (as described in DE102013217316, for example) and is usually indicated in mm²/10 min.

In a seventh preferred embodiment, cosmetic preparations are exemplified as contemplated herein in that the oil mixture has a refractive index (at 20° C.) in the range of from about 1,420 and about 1,430 and a spreadability (at 25° C.) in the range of from about 14.5 cm²/5 min to about below about 16.0 cm²/5 min.

Oil mixtures with a spreading range from about 14.5 cm²/5 min to about below 16.0 cm²/5 min (25° c) and a refractive index in the range of from about 1,420 and about 1,430 are particularly suitable for use in cosmetic preparations for body care, in particular for use in antiperspirant preparations, as they are particularly good at ensuring the concealment of white residues of powdery ingredients, such as aluminum chlorohydrate, on textiles.

The oil mixture is particularly suitable for use in anhydrous preparations for personal care, in particular in anhydrous antiperspirant preparations.

In an eighth preferred embodiment, cosmetic preparations are exemplified as contemplated herein in that they are largely anhydrous.

As contemplated herein, the term “largely anhydrous” indicates that the cosmetic preparations contain from about 0 to a maximum of about 3 wt. %, preferably from about 0 to a maximum of about 2 wt. %, of free water relative to the total preparation.

The content of water of crystallization, hydration water or similarly molecularly bound water that can be contained in the ingredients used, in particular in any included antiperspirant ingredients, is not free water in the sense of the present application.

As already stated, the preparations for personal care as contemplated herein are particularly suitable as an antiperspirant preparation.

Consequently, in a ninth preferred embodiment, the preparations as contemplated herein are packaged as an antiperspirant preparation, preferably as an anyhydrous antiperspirant stick or as an anyhydrous antiperspirant aerosol.

Antiperspirants usually contain an antiperspirant ingredient. Consequently, in a further preferred embodiment, it is beneficial if the cosmetic preparations as contemplated herein also contain an antiperspirant ingredient, preferably an antiperspirant aluminum salt.

A second subject matter of the present disclosures is a cosmetic antiperspirant preparation that contains

at least one ester a) that is formed from at least one linear or branched, saturated or unsaturated mono-, di- or tricarboxylic acid with 3 to 30 carbon atoms, which can contain one or more hydroxyl groups, and at least one linear or branched, saturated or unsaturated alcohol with from 1 to 30 carbon atoms, with a weight proportion of from about 10 to about 50 wt. % of the total weight of the preparation,

at least one cyclic polydimethylsiloxane b) with a weight proportion of from about 5 to about 30 wt. % of the total weight of the preparation,

at least one linear polydimethylsiloxane c) that has a kinematic viscosity of less than about 20 cSt at 25° C., with a weight proportion of from about 1 to about 20 wt. % of the total weight of the preparation, and

at least one antiperspirant aluminum salt d) with a weight proportion of from about 10 to about 40 wt. % of the total weight of the preparation.

In this embodiment, antiperspirant preparations that contain

from about 10 to about 50 wt. %, preferably from about 15 to about 45 wt. %, of 2-ethylhexyl laurate, 2-ethylhexyl myristate, 2-ethylhexyl palmitate, 2-ethylhexyl cocoate, 2-ethylhexyl stearate, 2-ethylhexyl isostearate, hexyldecyl laurate, hexyldecyl stearate, isopropyl myristate, isopropyl palmitate, isopropyl stearate, isooctyl stearate, isononyl isononanoate, isononyl stearate, isotridecyl nonanoate, 2-octyldodecyl palmitate, isocetyl stearate and/or mixtures of these esters, preferably 2-ethylhexyl palmitate and isopropyl myristate,

from about 0.5 to about 10 wt.-%, preferably from about 1 to about 6 wt.-%, of at least one methyl-, ethyl-, n-propyl-, isopropyl-, n-butyl-, 2-ubtyl- or tertiary butyl ester of glycolic acid, lactic acid, malic acid, tartaric acid, citric acid, oxalic acid, malonic acid, succinic acid, glutaric acid and/or adipic acid, preferably triethyl citrate

from about 5 to about 30 wt. %, preferably from about 10 to about 25 wt. %, of hexamethylcy clotrisiloxane, octamethylcy clotetrasiloxane, decamethylcyclopentasiloxane and/or dodecamethylcyclohexasiloxane, preferably decamethylcyclopentasiloxane,

from about 1 to about 20 wt. %, preferably from about 5 to about 15 wt. %, of a polydimethylsiloxane known by the INCI name dimethicone that has a kinematic viscosity of less than about 20 cSt at 25° C., preferably less than 10 cSt, and

from about 10 to about 40 wt. %, preferably from about 15 to about 25 wt. %, of at least one antiperspirant aluminum salt.

Preferred antiperspirant ingredients as contemplated herein are water-soluble astringent inorganic and organic salts of aluminum, zirconium and zinc or any mixtures of these salts. Most preferred antiperspirant ingredients are selected from aluminum chlorohydrate complexes, for example aluminum sesquichlorohydrate, aluminum chlorhydrate propylene glycol (PG) or aluminum chlorohydrate polyethylene glycol (PEG), aluminum sesquichlorohydrate PG or PEG, aluminum PG dichlorhydrate or aluminum PEG dichlorohydrate, aluminum hydroxide, and are also selected from aluminum zirconium chlorhydrate complexes such as aluminum zirconium trichlorohydrate, aluminum zirconium tetrachlorohydrate, aluminum zirconium pentachlorohydrate, aluminum zirconium octachlorhydrate, aluminum zirconium chlorohydrate glycine complexes such as aluminum zirconium trichlorohydrex glycine, aluminum zirconium tetrachlorohydrex glycine, aluminum zirconium pentachlorohydrex glycine, aluminum zirconium octachlorohydrex glycine, potassium aluminum sulfate (KAI(S0₄)₂ 0.12 H₂O, alum), aluminum undecylenoyl collagen amino acids, sodium aluminum lactate+aluminum sulfate, sodium aluminum chlorohydroxy lactate, aluminum bromide hydrate, aluminum chloride, complexes of zinc and sodium salts, complexes of lanthanum and cerium, aluminum salts of lipoamino acids, aluminum sulfate, aluminum lactate, aluminum chlorohydroxy allantoinate, sodium aluminum chlorhydroxy lactate, zinc chloride, zinc sulfocarbolate, zinc sulfate and zirconium chlorohydrate.

As contemplated herein, “water solubility” refers to a solubility of at least about 5 wt. % in water at 20° C., i.e. quantities of at least 5 g of the antiperspirant ingredient are soluble in 95 g of water at 20° C. The active antiperspirant ingredients can be used as aqueous solutions.

Most preferred antiperspirant preparations as contemplated herein are exemplified in that they contain at least one antiperspirant ingredient, preferably selected from astringent aluminum salts, in particular aluminum chlorohydrate, aluminum sesquichlorohydrate and/or aluminum chloride, in a total quantity of from about 10 to about 40 wt. %, preferably of from about 12.5 to about 30 wt. % and most preferably from about 15 to about 25 wt. %, wherein the quantity data are relative to the total weight of the antiperspirant preparation.

In a further preferred embodiment, the antiperspirant preparations of the second subject matter of the present disclosure can also contain at least one deodorizing ingredient, which can be selected from odor absorbers, deodorizing ion exchangers, germ-inhibiting substances, prebiotic ingredients and inhibitors of enzymes responsible for the decomposition of perspiration or, most preferably, combinations of these ingredients.

Silicates can be used as preferred odor absorbers that also simultaneously enhance the rheological properties of the antiperspirant preparations. The most preferred silicates include, above all, sheet silicates, and, among these, in particular montmorillonite, kaolinite, illite, beidellite, nontronite, saponite, hectorite, bentonite, smectite and talcum. Examples of other most preferred odor absorbers are zeolites, zinc ricinoleate, cyclodextrins, certain metal oxides, such as aluminum oxide, as well as chlorophyll. Odor absorbers can be used in the antiperspirant preparations, preferably in a quantity of from about 0.1 to about 10 wt. %, more preferably of from about 0.5 to about 7 wt. % and most preferably of from about 1 to about 5 wt. %, relative to the total weight of the antiperspirant preparations in each case.

Preferred antiperspirant preparations are exemplified in that they contain at least one odor absorber, preferably a silicate.

Germ-inhibiting or antimicrobial substances also include those substances that reduce the number of skin germs involved in the development of odors or inhibit their growth.

These germs include, among others, various species from the group of staphylococci (e.g. staphylococcus hominis), the group of corynebacteria (e.g. corynebacterium xerosis, corynebacterium CDCG2), anaerococci (e.g. anaerococcus octavius) and micrococci. Preferably, the mixtures of fragrances Protectate HR and Protectate MOD 2 produced by Symrise can be used as germ-inhibiting or antimicrobial substances.

The mixture of fragrances Protectate HR produced by Symrise contains from about 25 to about 50 wt. % of phenoxyethanol, from about 5 to about 10 wt. % of 2-methyl-5-phenylpentan-1-ol with the trivial name Rosaphen, about 34 to about 70 wt. % of 2-benzylheptan-1-ol with the trivial name Jasmol, from about 1 to about 5 wt. % of 4-methoxybenzyl alcohol (anisyl alcohol) and from about 0.01 to about 1 wt. % of 5-methyl-2-isopropylphenol (thymol). The mixture of fragrances Protectate MOD 2 produced by Symrise contains from about 25 to about 45 wt. % of phenoxyethanol, from about 5 to about 10 wt. % of 2-methyl-5-phenylpentan-1-ol and from about 45 to about 70 wt. % of 2-benzylheptan-1-ol.

Organic halogen compounds and halides, quaternary ammonium cations, a number of plant extracts and zinc compounds are also preferred germ-inhibiting or antimicrobial substances. This includes triclosan, chlorhexidine and chlorhexidine gluconate, 3,4,4′-trichlorocarbanilide, bromchlorophen, dichlorophen, chlorothymol, chloroxylenol, hexachlorophene, dichloro-m-xylenol, dequalinium chloride, domiphen bromide, ammonium phenolsulfonate, benzalkonium halide, benzalkonium cetyl phosphate, benzalkonium saccharinate, benzethonium chloride, cetylpyridinium chloride, lauryl pyridinium chloride, lauryl isoquinolinium bromide, methylbenzethonium chloride. Furthermore, phenol, phenoxyethanol, disodium dihydroxyethyl sulfosuccinylundecylenate, sodium bicarbonate, zinc lactate, sodium phenolsulfonate and zinc phenolsulfonate, ketoglutaric acid, terpenic alcohols such as the most preferred farnesol, copper chlorophyllin, α-monoalkyl glycerin ether with a branched or linear saturated or unsaturated, possibly hydroxylated C₆-C₂₂ alkyl radical, most preferably α-(2-Ethyl-hexyl)glycerinether, commercially available as Sensiva® SC 50 (produced by Schülke & Mayr), carboxylic acid esters of mono-, di- and triglycerines (e.g. glycerol monolaurate, diglycerin monocaprinate), lantibiotics and plant extracts (e.g. green tea and elements of lime blossom oil,) are preferred deodorizing substances.

Other preferred deodorizing substances are selected from prebiotic substances, which are those ingredients that only or at least predominantly inhibit the odor-forming germs in the skin microflora, but not the desirable, i.e. not odor-forming, germs that belong to a healthy skin microflora. This particularly includes those substances that are disclosed as prebiotic in published patent applications DE 10333245 and DE 10 2004 011 968, including conifer extracts in particular from the group of Pinaceae, and plant extracts from the group of Sapindaceae, Araliaceae, Lamiaceae and Saxifragaceae, in particular extracts of Picea spp., Paullinia sp., Panax sp., Lamium album or Ribes nigrum and mixtures of these substances. Further preferred deodorizing substances are selected from the germ-inhibiting perfume oils and Deosafe perfume oils, which are available from Symrise (formerly Haarmann and Reimer). Deodorizing enzyme inhibitors are those substances that inhibit the enzymes responsible for the decomposition of perspiration, in particular arylsulfatase, β-glucuronidase, aminoacylase, ester-cleaving lipases and lipoxygenase, wherein zinc glycinate is preferred.

The above-mentioned deodorizing substance(s) can be contained in the antiperspirant preparations of the second subject matter of the present disclosure, preferably in a total quantity of from about 0.1 to about 10 wt. %, more preferred from about 0.2 to about 7.5 wt. %, most preferred from about 0.3 to about 5 wt. % and especially preferred from about 0.5 to about 3.0 wt. %, relative to the total weight of the preparation.

Furthermore, preferred antiperspirant preparations as contemplated herein are exemplified in that they contain at least one encapsulated and/or at least one non-encapsulated fragrance.

Preferably, the encapsulation of fragrances can be selected so that it includes at least one water-soluble encapsulation material. When exposed to moisture, here in particular skin moisture or sweat, the water-soluble encapsulation material opens a certain time after application, and the encapsulated fragrance and any other encapsulated substances, such as skin-cooling agents, are released with a time delay after application.

Encapsulated and non-encapsulated fragrances, such as perfume oils or mixtures of perfume oils, can be identical or different. Most preferred antiperspirant preparations are exemplified in that they contain at least one encapsulated and at least one non-encapsulated fragrance, which are different from one another.

Preferred antiperspirant preparations are exemplified in that they contain at least one non-encapsulated fragrance in a total quantity of from about 0.1 to about 3 wt. %, preferably from about 0.2 to about 1.5 wt. %, and most preferably from about 0.4 to about 1 wt. %, relative to the total weight of the aerosol composition in each case.

Further preferred antiperspirant preparations are exemplified in that they at least one encapsulated fragrance in a total quantity of from about 0.01 to about 2 wt. %, preferably from about 0.1 to about 1.0 wt. %, and most preferably from about 0.25 to about 0.5 wt. %, relative to the total weight of the aerosol composition in each case.

Fragrance compounds such as synthetic products based on esters, ethers, aldehydes, ketones, alcohols and hydrocarbons are most preferably used as fragrances or perfume oils. The preferred phenolic fragrance compounds include, for example, carvacrol. Preferred fragrance compounds based on esters include, for example, benzyl acetate, methyl anthranilate, ortho-tert-butyl cyclohexyl acetate, p-tert-butyl cyclohexyl acetate, diethyl phthalate, nonanediol-1,3-diacetate, isononyl acetate, isononyl formate, phenylethyl phenylacetate, phenoxyethyl isobutyrate, linalyl acetate, dimethyl benzyl carbinyl acetate, phenylethyl acetate, linalyl benzoate, benzyl formate, ethyl methylphenylglycinate, allyl cyclohexyl propionate, styrallyl propionate, benzyl salicylate, ethyl salicylate, isoamyl salicylate, hexyl salicylate and 4-nonanolide. The preferred ethers include, for example, benzyl ethyl ether; the preferred aldehydes include linear alkanes with 8 to 18 carbon atoms, citral, citronellal, citronellyl oxyacetaldehyde, cyclamen aldehyde, hydroxycitronellal, lilial and bourgeonal; the preferred ketones include, 6-acetyl-1,1,3,4,4,6-hexamethyltetra-hydronaphthalin, para-t-amyl cyclohexanone, 2-n-heptyl cyclopentanone, β-methyl naphthyl ketone and ionones a-isomethyl ionone and methyl cedryl ketone; the preferred alcohols include cinnamyl alcohol, anethole, citronellol, dimyrcetol, eugenol, geraniol, linalool, phenethyl alcohol and terpineol; the preferred hydrocarbons include 1,3,4,6,7,8-hexahydro-4,6,6,7,8,8-hexamethyl-cyclopenta-a-2-benzopyran, hydroxymethyl isopropylcyclopentane, 3-a-methyl dodecahydro-6,6,9a-trimethylnaphtho-2(2,1-b)furan, isobutyl quinoline as well as terpenes and balms. Most preferably, mixtures of different fragrances are used that, together, produce a pleasant scent. Most preferred perfume oils can also contain natural mixtures of fragrances, such as those that are available from plant or animal sources, e.g. pine, citrus, jasmine, ylang ylang, rose or lily oil. Less volatile essential oils that are mostly used as aroma components are also most preferred as perfume oils, e.g. sage oil, chamomile oil, melissa oil, mint oil, cinnamon leaf oil, lime blossom oil, juniper berry oil, vetiver oil, oliban oil, galbanum oil, laudanum oil, clove oil, isoeugenol, thyme oil, rose oil, bergamot oil and geranium oil.

Preferred capsule materials are water-soluble polymers such as starches, physically and/or chemically modified starches, cellulose derivatives, such as carboxymethyl cellulose, methyl cellulose, hydroxyethyl cellulose or hydroxypropyl methylcellulose, carrageenans, alginates, maltodextrins, dextrins, plant gums, pectins, xanthans, polyvinyl acetate and polyvinyl alcohol, polyvinylpyrrolidone, polyamides, polyester and homo- and copolymers of monomers, selected from acrylic acid, methacrylic acid, maleic acid, fumaric acid, itaconic acid and the esters and salts of these acids, as well as any mixtures of these polymers.

Most preferred capsule materials are chemically modified starches, in particular aluminum starch octenylsuccinate, e.g. the commercial product Dry Flo Plus produced by National Starch, or sodium starch octenylsuccinate, e.g. the commercial product Tylose H 10 produced by Clariant, and also carboxymethyl cellulose, carboxymethyl cellulose, methyl cellulose, hydroxyethyl cellulose and hydroxypropyl methylcellulose, as well as carrageens, alginates and maltodextrins, and any mixtures of these polymers.

Most preferred capsule materials are polymer blends, including of chemically modified starches and/or hydroxyethyl cellulose and a proportion of from about 0.2 to about 2 wt. % of alginates and/or carrageenans.

Encapsulation can take place according to the known procedure. Appropriate procedures are, for example, disclosed in K. Masters, “Spray Drying Handbook”, 3rd edition, John Wiley, 1979. In a most preferred encapsulation procedure, a water-based mixture is produced, containing approx. 20 to about 50 wt. % of the polymeric encapsulation material, approx. 0.1 to about 2.0 wt. % of an emulsifier, approx. 5 to about 20 wt. % of the perfume oil to be encapsulated and/or the skin-cooling agent to be encapsulated and approx. 40 to about 60 wt. % water. This mixture is homogenized and then spray-dried. The capsules loaded with active agent are thus obtained as a fine powder with a particle diameter of about 1 to about 150 μm, preferably from about 20 to about 80 μm, most preferably from about 5 to about 50 μm.

In another manufacturing process, microencapsulation takes place by employing coacervation, whereby the carrier of gelatine.

The capsule material, including of water-soluble polymers and a low content of emulsifiers, enables a reversible “reencapsulation” of encapsulated perfume oils and skin-cooling agents. Reencapsulation takes place in situ while the skin dries following a period of perspiration. Consequently, various, successive activities take place on the skin, without the user having to reapply the product as contemplated herein.

As contemplated herein, fragrance-free or non-perfumed antiperspirant preparations can also be preferred.

Preferred antiperspirant preparations of the second subject matter of the present disclosure are also exemplified in that they contain at least one suspension or thickening agents, preferably selected from hydrophobic clay minerals and fumed silica. Preferred hydrophobic clay minerals are montmorillonite, hectorite and bentonite, in particular disteardimonium hectorite and quaternium-18 hectorite. Commercially available thickening agents provide these hydrophobic clay minerals in the form of a gel in cyclomethicone and, if desired, an additional oil component, such as propylene carbonate. Further preferred thickening agents are fumed silica, e.g. the commercial products of the Aerosil® series produced by Degussa.

Preferably, the antiperspirant preparations of the second subject matter of the present disclosure can be produced as anhydrous stick preparations or as an anhydrous antiperspirant aerosol. Anhydrous antiperspirant aerosols can be beneficial for some applications. Antiperspirant aerosols suitable as contemplated herein are preferably packaged in commercially available aerosol cans. The cans can be made of tin or aluminum. Furthermore, the insides of the cans can be coated according to a most preferred embodiment, in order to minimize the risk of corrosion.

Preferably, the aerosol cans should be equipped with a suitable spray nozzle. Depending on the spray nozzle, flow rates (based on a completely full can) of from about 0.1 g/s to about 2.0 g/s are preferred.

A third subject matter of the present disclosure is the cosmetic use of a cosmetic preparation as a personal care product, in particular as an antiperspirant preparation, that has a reduced tendency to cause stain formation on textiles and/or is more easily washed out of textiles.

For preferred embodiments of use as contemplated herein, the cosmetic preparations for personal care and for antiperspirant preparations mentioned in the present disclosure apply mutatis mutandis.

The examples below should illustrate the present disclosure, without limiting it to these examples.

### EXAMPLES

### I. Proof of Efficacy

Different oil mixtures were produced (see Table 1). 75 μl of each of the oil mixtures was applied to blue cotton textile. The wetted area was measures after 5 minutes and is given as a spreadability value in cm² in Table 1. The respective textile was then sprayed with a 25% solution of aluminum chlorohydrate, dried and washed at 40° C. using a commercially available detergent (Persil Universal). Once dry, the stain formation on the respective textile was visually assessed by experts and red using a 4-point reference scale (1=very weak stain; 4=very strong stain). The optical refractive index of the respective oil mixture was determined at 20° C.

TABLE 1

| | | | | | | | |
|---|---|---|---|---|---|---|---|
| 2-ethylhexyl palmitate [wt. %] | 76.00 | 40.51 | 61.28 | 80.00 | 40.00 | 67.63 | 72.65 |
| Isopropyl myristate [wt. %] | 11.99 | 5.76 | 0.00 | 1.00 | 13.50 | 1.32 | 12.78 |
| Triethyl citrate [wt. %] | 1.01 | 4.24 | 5.00 | 0.00 | 2.35 | 2.79 | 2.89 |
| Cyclopentasiloxane [wt. %] | 0.00 | 36.00 | 26.55 | 14.85 | 36.00 | 28.25 | 11.67 |
| Dimethicone 5 cSt [wt. %] | 11.00 | 13.50 | 6.63 | 4.16 | 8.15 | 0.00 | 0.00 |
| Spreadability value [cm²/5 min] | 13.4 | 15.1 | 14.5 | 13.8 | 15.2 | 13.5 | 15.5 |
| Stain formation | 3 | 1 | 2.5 | 2.5 | 1.5 | 3 | 2.5 |
| Refractive index | 1,440 | 1,423 | 1,431 | 1,438 | 1,425 | 1,434 | 1,440 |

### II. Application Examples

### a) Antiperspirant Aerosols (Quantity Data in wt. %)

| | 1.1 | 1.2 | 1.3 |
|---|---|---|---|
| Cyclopentasiloxane (Xiameter ®¹ PMX-0245) | 14.0 | 21.8 | 19.5 |
| Isopropyl myristate | 10.0 | 10.0 | 7.1 |
| 2-ethylhexyl palmitate | 30.0 | 30.0 | 30.8 |
| Dimethicone 5 cSt (Xiameter ®² PMX-200 Fluid | 10.0 | 10.0 | 9.5 |
| 5 cSt) | | | |
| Triethyl citrate | 4.0 | 4.0 | 2.6 |
| Bentone ®³ 38 V CG | 2.5 | 2.5 | 2.5 |
| Propylene carbonate | 1.0 | 1.0 | 1.0 |
| Aluminum chlorohydrate (AACH ®⁴ 7172) | 23.5 | 15.7 | 21.9 |
| Perfume | 5.0 | 5.0 | 5.0 |

Formulations 1.1, 1.2 and 1.3 are filled into in aerosol cans with the propellant propane/butane in a weight ratio of 1:4 (15/85).

The following commercial products were used:
1 INCI name: Cyclomethicone (96% AS); Xiameter (Dow Corning)
2 INCI name: Dimethicone 5 cSt (100% AS), Xiameter (Dow Corning)
3 INCI name: Disteardimonium hectorite; Elementis Specialties
INCI name: Aluminum chlorohydrate (from about 76.5- about 93.5% AS); Summit Research Labs, Inc.

### b) Antiperspirant Sticks (Quantity Data in wt. %)

| | 2.1 | 2.2 | 2.3 | 2.4 | 2.5 |
|---|---|---|---|---|---|
| Aluminum zirconium trichlorohydrex | 17.8 | | 17.8 | 17.8 | 23.0 |
| gly (AAZG ®⁵ 531) | | | | | |
| Aluminum chlorohydrate | | 17.8 | | | |
| (Microdry ®⁶ 3115) | | | | | |
| Stearyl alcohol (Lanette ®⁷18) | 19.5 | 19.5 | 19.5 | 19.5 | 19.5 |
| Cetyl alcohol (Lorol ®⁸ 20) | 0.4 | 0.4 | 0.4 | 0.4 | 0.4 |
| Arachidyl alcohol (Nacol ®⁹ 20-95) | 0.1 | 0.1 | 0.1 | 0.1 | 0.1 |
| PPG-14 butyl ether (Ucon Fluid ®¹ | 6.0 | 6.0 | 6.0 | 6.0 | 6.0 |
| AP) | | | | | |
| Myristyl myristate (Crodamol ®² | 2.0 | 2.0 | | 2.0 | 2.0 |
| MM) | | | | | |
| Talcum | 5.5 | 5.5 | 5.5 | 1.5 | |
| Hydrogenated castor oil | 2.8 | 2.8 | 2.8 | 2.8 | 2.8 |
| (Cutina ®² HR) | | | | | |
| Cyclopentasiloxane (Xiameter ®¹ | 16.5 | 16.5 | 18.5 | 16.5 | 16.7 |
| PMX-0245) | | | | | |
| Isopropyl myristate | 4.3 | 4.3 | 4.3 | 4.3 | 4.3 |
| 2-ethylhexyl palmitate | 17.0 | 17.0 | 17.0 | 17.0 | 17.0 |
| Dimethicone 5 cSt (Xiameter ®² | 5.6 | 5.6 | 5.6 | 5.6 | 5.6 |
| PMX-200 Fluid 5 cSt) | | | | | |
| Triethyl citrate | 1.6 | 1.6 | 1.6 | 1.6 | 1.6 |
| Perfume | 1.0 | 1.0 | 1.0 | 1.0 | 1.0 |

The following commercial products were used:
5 INCI name: Aluminum zirconium octachlorohydrex GLY; Summit Research
6 INCI name: Aluminum chlorohydrate; Reheis
7 INCI name: Stearyl alcohol; BASF
8 INCI name: Cetyl alcohol; BASF
9 INCI name: Arachidyl alcohol; Sasol
10 INCI name: PPG-14 butyl ether; Dow
11 INCI name: Myristyl myristate; Croda
12 INCI name: Hydrogenated castor oil; BASF

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic preparation for personal care, comprising, (relative to the total weight of the cosmetic preparation, an oil mixture in a total quantity of from about 40 to about 99 wt. %, wherein the oil mixture comprises:
a) at least one ester that is formed from at least one linear or branched, saturated or unsaturated mono-, di- or tricarboxylic acid with from 3 to 30 carbon atoms, which comprises one or more hydroxyl groups, and at least one linear or branched, saturated or unsaturated alcohol with 1 to 30 carbon atoms,
b) at least one cyclic polydimethylsiloxane, and
c) at least one linear polydimethylsiloxane that has a kinematic viscosity of less than about 20 cSt at 25° C.

2. Cosmetic preparation according to claim 1, wherein the oil mixture comprises:
at least one ester a-1) selected from 2-ethylhexyl laurate, 2-ethylhexyl myristate, 2-ethylhexyl palmitate, 2-ethylhexyl cocoate, 2-ethylhexyl stearate, 2-ethylhexyl isostearate, hexyldecyl laurate, hexyldecyl stearate, isopropyl myristate, isopropyl palmitate, isopropyl stearate, isooctyl stearate, isononyl isononanoate, isononyl stearate, isotridecyl nonanoate, 2-octyldodecyl palmitate, isocetyl stearate and/or mixtures thereof, and
at least one ester a-2), selected from methyl-, ethyl-, n-propyl-, isopropyl-, n-butyl-, 2-ubtyl- or tertiary butyl esters of glycolic acid, lactic acid, malic acid, tartaric acid, citric acid, oxalic acid, malonic acid, succinic acid, glutaric acid and/or adipic acid.

3. Cosmetic preparation according to claim 1, wherein the oil mixture comprises at least two different esters from group of esters a-1).

4. Cosmetic preparation according to claim 3, wherein the oil mixture comprises 2-ethylhexyl palmitate and isopropyl myristate as esters a-1).

5. Cosmetic preparation according to claim 1, wherein the oil mixture comprises a methyl or ethyl ester of tartaric acid, citric acid and/or oxalic acid as ester a-2).

6. Cosmetic preparation according to claim 1, wherein the oil mixture comprises at least one cyclic polydimethylsiloxane b) that is selected from hexamethylcyclotrisiloxane, octamethylcyclotetrasiloxane, decamethylcyclopentasiloxane and/or dodecamethylcyclohexasiloxane.

7. Cosmetic preparation according to claim 1, wherein the oil mixture comprises at least one linear polydimethylsiloxane c) that has a kinematic viscosity of less than about 15 cSt at 25° C.

8. Cosmetic preparation according to claim 1, wherein the oil mixture comprises:
from about 35 to about 70 wt. % relative to the total weight of the oil mixture of at least one ester a),
from about 20 to about 40 wt. % relative to the total weight of the oil mixture of at least one cyclic polydimethylsiloxane b), and
from about 5 to about 20 wt. % relative to the total weight of the oil mixture of at least one linear polydimethylsiloxane c) that has a kinematic viscosity of less than about 20 cSt at 25° C.

9. Cosmetic preparation according to claim 1, wherein the oil mixture has a refractive index (at 25° C.) of about 1,420 to about 1,430 and a spreadability (at 25° C.) of from about 14.5 cm²/5 min to about below 16.0 cm²/5 min.

10. Cosmetic preparation according to claim 1, wherein the cosmetic preparation is substantially anhydrous.

11. Cosmetic preparation according to claim 1, wherein the cosmetic preparation additionally comprises at least one antiperspirant ingredient including an antiperspirant aluminum salt d).

12. Cosmetic preparation according to claim 1, wherein the cosmetic preparation is produced as an antiperspirant preparation.

13. Cosmetic antiperspirant preparation, comprising
a) at least one ester a) that is formed from at least one linear or branched, saturated or unsaturated mono-, di- or tricarboxylic acid with from 3 to 30 carbon atoms, which comprises one or more hydroxyl groups, and at least one linear or branched, saturated or unsaturated alcohol with from 1 to 30 carbon atoms, with a weight proportion of from about 10 to about 50 wt. % of the total weight of the preparation,
b) at least one cyclic polydimethylsiloxane b) with a weight proportion of from about 5 to about 30 wt. % of the total weight of the preparation,
c) at least one linear polydimethylsiloxane c) that has a kinematic viscosity of less than about 20 cSt at 25° C., with a weight proportion of from about 1 to about 20 wt. % of the total weight of the preparation, and
d) at least one antiperspirant aluminum salt d) with a weight proportion of from about 10 to about 40 wt. % of the total weight of the preparation.

14. Cosmetic antiperspirant preparation according to claim 13, wherein the cosmetic antiperspirant preparation comprises:
a) from about 10 to about 50 wt. %, of 2-ethylhexyl laurate, 2-ethylhexyl myristate, 2-ethylhexyl palmitate, 2-ethylhexyl cocoate, 2-ethylhexyl stearate, 2-ethylhexyl isostearate, hexyldecyl laurate, hexyldecyl stearate, isopropyl myristate, isopropyl palmitate, isopropyl stearate, isooctyl stearate, isononyl isononanoate, isononyl stearate, isotridecyl nonanoate, 2-octyldodecyl palmitate, isocetyl stearate and/or mixtures thereof,
b) from about 0.5 to about 10 wt. %, of at least one methyl-, ethyl-, n-propyl-, isopropyl-, n-butyl-, 2-ubtyl- or tertiary butyl ester of glycolic acid, lactic acid, malic acid, tartaric acid, citric acid, oxalic acid, malonic acid, succinic acid, glutaric acid and/or adipic acid,
c) from about 5 to about 30 wt. %, of hexamethylcyclotrisiloxane, octamethylcyclotetrasiloxane, decamethylcyclopentasiloxane and/or dodecamethylcyclohexasiloxane,
d) from about 1 to about 20 wt. %, of a polydimethylsiloxane that has a kinematic viscosity of less than about 20 cSt at 25° C., and
e) from about 10 to about 40 wt. %, of at least one antiperspirant aluminum salt.

15. Cosmetic use of a preparation according to claim 1 as a personal care product that has a reduced tendency to cause stain formation and/or is more easily washed out of textiles.
